(19) United States
(12) Patent Application Publication
(10) Pub. No.: US 2018/0264245 A1
(43) Pub. Date: Sep. 20, 2018

(54) Skin Care Applicator

(71) Applicant: The Procter & Gamble Company, Cincinnati, OH (US)

(72) Inventors: Jeffrey David EDWARDS, Perth (AU); Matthew James MCILDOWIE, Perth (AU)

(21) Appl. No.: 15/922,532

(22) Filed: Mar. 15, 2018

Related U.S. Application Data
(60) Provisional application No. 62/471,977, filed on Mar. 16, 2017.

(51) Int. Cl. A61M 37/00; A61N 2/00; A61H 23/02

(52) U.S. Cl. CPC A61M 37/0092; A61N 2/002; A61M 35/003; A45D 2200/1036; A61H 2201/105; A61H 23/0263

(57) ABSTRACT

An applicator for a skin care product is provided, together with a cosmetic skin care product comprising an applicator and a skin care composition, and a method of using the applicator to regulate a skin condition. The applicator has a substrate with a magnetic array printed thereon, the magnetic array having at least one layer of one or more dipolar pairs of alternating magnetic poles. The applicator further has a vibration source. The magnetic field strength generated by the magnetic array is at its maximum at the poles. When activated, the vibration source causes side to side movement of the applicator, meaning that the full magnetic field is experienced by all diamagnetic materials to which the applicator is applied.

### FIELD OF THE INVENTION

The present invention relates to applicators for skin care products that provide enhanced penetration of a skin care active into skin, and to a method for enhancing delivery of a skin care active into skin. Specifically, the present invention relates to applicators that combine benefits of diamagnetism and vibration to optimize penetration of skin care actives into skin.

### BACKGROUND OF THE INVENTION

Topical skin care compositions containing actives that provide benefits to skin are well known. For example, Vitamin B3 compounds, particularly niacinamide are known to provide measurable skin regulating benefits. Topical niacinamide is known to help regulate the signs of skin aging, by reducing the visibility of the fine lines, wrinkles, and other forms of uneven or rough surface texture associated with aged or photo-damaged skin. These compounds have also been found useful in reducing the overall oiliness of skin. Likewise, peptides (e.g., di-, tri-, tetra- and pentapeptides) and their derivatives, known for use in regulating a variety of skin conditions, typically need to penetrate skin to provide the desired benefit. In one particular example, the peptide derivative palimitoyl-lysine-threonine-threonine-lysine-serine (“pal-KTTKS”) is used in skin care compositions to improve the signs of skin aging.

However, effective and optimal delivery of skin care actives, such as niacinamide or pal-KTTKS, into skin is an ongoing challenge. Typically, active agents with skin care benefits are introduced to skin via topical application of, for example, creams, lotions and essences. However, the actual and perceived benefits of skin care actives are largely dependent on the amount of skin care active that penetrates the top layer of skin and the depth to which it penetrates. There are various factors that limit the amount of active agent that can penetrate skin, and at present there is little control over the positioning and residency of the active agents following penetration into skin.

The amount of active agent provided in a skin care composition can be increased in various ways, for example, by increasing the amount of active agent in the skin care composition. However, this often leads to compositions that do not have a good sensory feel, increased formulation challenges, stability issues and increased manufacturing costs.

One approach to improving the efficacy of a skin care active is to use chemical penetration enhancers to facilitate changes in skin permeability, allowing enhanced penetration of the skin care active. However, the use of chemical penetration enhancers can be problematic due to unknown interaction with the active agent and the potential for adverse side effects such as irritation of skin and mucosal surfaces.

Mechanical approaches to increasing skin penetration of actives have also been explored. For example, one such approach known as iontophoresis utilizes an electrical energy gradient to accelerate a charged active agent(s) across the skin (or other barrier). An example of a device that uses iontophoresis is described in U.S. Pat. No. 7,137,965. However, iontophoresis is only suitable for specific active agents with certain ionic structures and can be injurious to certain dermal barriers due to exchange ion degradation. Additionally, iontophoresis requires the use of intimate electrical contact and adhesive electrodes, which are not suitable for all target surfaces or barriers.

Other techniques for creating mobility and/or direction in the movement of active agent(s) include magneto kinetics and magneto-phoresis. However, these techniques have been difficult to implement due to poor performance, high hardware and energy requirements, and cost. An example of a device that utilizes magnetophoresis is described in US 2009/0093669. While these methods claim to increase the amount of penetration of skin care actives into skin, they still do not provide enhanced penetration in a controlled manner—both in terms of amount of penetration and depth of penetration.

In another example of a device designed to effectively deliver skin care actives, WO 2011/156869 discloses a method of delivering a skin care agent through a dermal barrier using one or more displaced dipolar magnetic elements.

However, there is still more that can be done to enhance and optimize penetration of skin care actives into skin.

### SUMMARY OF THE INVENTION

According to a first aspect of the invention, there is provided an applicator for a skin care product, comprising a) a substrate with a magnetic array printed thereon, said magnetic array having at least one layer of one or more dipolar pairs of alternating magnetic poles; and b) a vibration source.

Both vibration and use of a dipolar magnetic array are independently known to enhance penetration of skin care actives into skin. Use of vibration in an applicator that is brought into contact with a keratinous surface, such as skin, is known to enhance penetration of skin care actives applied with the applicator or shortly after use of the applicator, through physical displacement of the skin. Use of a dipolar magnetic array is known to enhance penetration of skin care actives into skin via the diamagnetic force applied by the magnetic array to diamagnetic particles in a skin care composition. The present inventors have surprisingly discovered a synergy between use of a vibrating applicator that has a built in dipolar magnetic array. Without being bound by theory, it is thought that the diagmagnetic force between the applicator and the diamagnetic particles is strongest at the peaks of magnetic flux generated by the dipolar magnets. By rapidly moving the applicator (through the vibratory movement), a greater number of diamagnetic particles “see” the peak magnetic flux in a shorter period of time (vs manual movement of a magnetic applicator over skin). Thus, combined with the physical benefit of using a vibrating applicator, the resultant penetration into skin of a skin care active applied to skin with an applicator of the present invention is significantly greater.

The magnetic poles may be located a distance x (mm) apart, wherein x is at least 1. In embodiments, the vibration source is configured to vibrate with an amplitude of vibration at least half of x.

The position of the alternating poles marks the point at which the magnetic field strength (/magnetic flux) is at its maximum. Thus, the distance between poles is equal to the pitch between adjacent peaks of magnetic field strength. By having an amplitude of vibration of at least half the pitch of adjacent peaks of magnetic flux it is possible to ensure that nearly all diamagnetic particles experience the maximum magnetic flux.

In embodiments, the vibration source may have an amplitude of vibration of up to 1 mm, 1.5 mm or 2 mm. The vibration source may have a minimum amplitude of 0.1 mm, 0.2 mm, 0.4 mm or 0.5 mm preferably, the vibration source has an amplitude of about half the pitch between adjacent peaks of the dipolar magnet. Where the amplitude of vibration is about half the pitch between adjacent peaks, in theory all diamagnetic particles should experience the maximum diamagnetic force at some point and, preferably, repeatedly.

The vibration source may generate vibration of frequency lying in the range of from 0.5, 1, 10 or 50 hertz (Hz) to 200, 300, 500 or 1000 hertz (Hz). Increasing the frequency helps to ensure that all diamagnetic particles receive the same amount of magnetic force from the magnetic array. However, if the frequency is too high, the feel on the skin for a user may be unpleasant.

The vibration may be produced intermittently or continuously.

Typically, the vibration source comprises a motor, e.g., a disk-shaped or pancake motor, driving an off-center fly-weight in rotation. The speed of rotation of the motor may lie in the range 2500 revolutions per minute (RPM), 4500 RPM or 6500 RPM to 7000 RPM, 10000 RPM, 12500 RPM or 15000 (RPM), preferably in the range 4500 RPM to 10000 RPM.

The use of a motor driving an off-center fly-weight in rotation makes it possible to generate vibration of amplitude that is relatively small and of frequency that is relatively high.

Alternatively and/or additionally, the vibration source may be piezoelectric, electromechanical or eccentric. The vibration source may comprise a motor driving, in rotation, a toothed wheel that is in contact with an elastically-deformable blade, like a rattle.

The voltage may lie in the range of 1.3 volts (V) to 9V, for example. The vibration source may include an electricity source, such as 1.5V button battery. The use of a button battery may be advantageous to provide a compact device. When using a button battery and a disk-shaped motor, the battery and the motor may be face-to-face, side-by-side, or the face of one may face the edge of the other.

The applicator further comprises a power source for activation of the vibration source. In embodiments, the vibration source may be activated by a simple on/off switch. Alternatively, the vibration may be activated by one or more sensors, for example, proximity sensors that initiate the vibration upon contact with skin or pressure sensors that initiate the vibration upon increased pressure in the handle portion by a user. In embodiments, power for the vibration is activated upon completion of a circuit. In this embodiment, the applicator tip is formed of electrically conductive metal, and the base of the handle is formed of electrically conductive metal. If a user holds the applicator at the base of the handle on the metal area, the vibration source will be activated when the metal applicator tip is brought into contact with another body part. In this respect, the circuit is completion when the tip touches the skin surface to which the product is to be applied, and the user grasps the metal portion at the base of the handle. The contacts are part of a touch switch circuit that activates a vibration motor upon detecting a current path.

Alternatively and/or additionally, the applicator preferably includes a control member for controlling operation of the vibration source. The control member may be a simple on/off switch or trigger, operated by the user. The device may include a control member for controlling operation of the vibration source. The control member may be triggered by pressing it. The control member may be disposed in such a manner that it is pressed when the user takes hold of the device. The control member may operate upon completion of a circuit where

The vibration produced may be oriented substantially parallel to a longitudinal axis of the applicator. In a variant, the vibration may be substantially perpendicular to the longitudinal axis of the applicator.

The tip of the applicator may be stationary relative to the handle portion or it may be movable relative to the handle portion.

In embodiments, the magnetic array of the applicator comprises a first layer of alternating magnetic poles at least 1 mm apart, and a first layer magnetic field strength of between 12 mT and 30 mT. This results in a sine wave of flux density with peaks of magnetic field strength located at least 1 mm apart as seen by the diamagnetic particle. In this respect, diamagnetic particles see positive and negative flux equally. Thus, the range of magnetic flux experienced by a diamagnetic particle ranges from 0 to maximum magnetic field strength.

The magnetic array may further comprise a second layer of one or more dipolar pairs of alternating magnetic poles offset from the first layer at an angle of between 1° and 179°, the second layer of magnetic poles having a second layer pitch of between 1 mm and 3.5 mm, and a second layer magnetic field strength of between 8 mT and 24 mT, wherein the second layer magnetic field strength is less than or equal to the first layer magnetic field strength. Such a bi-directional magnetic array provides a more complex profile of magnetic field strength induced in the skin care active. Poles of the first and second layer constructively and destructively interfere with one another to reduce the areas of minimum magnetic flux density and ineffectual magnetic field strength.

The pitch of the second layer may be equal to or less than the pitch of the first layer. Additionally or alternatively, the overall magnetic field strength of the second layer is equal to or less than the overall magnetic field strength of the first layer. Typically the first layer is used to determine the maximum magnetic field strength, while the second layer smoothes out the overall profile of the magnetic field.

The magnetic array has a proximal skin facing side and a distal side opposed thereto, wherein a magnetic return is provided at the distal side of the ferromagnetic substrate. The magnetic return is used to integrate the magnetic fields generated by each pole on that side of the substrate and reduces or eliminates the magnetic flux on that surface, thus directing the magnetic flux towards the skin facing side.

The method of constructing the magnetic array may involve separately magnetizing two layers of substrate with the two respective layers of poles and arranging the first and second layers of substrate in parallel such that the distal side of the second layer is adjacent the proximal side of the first layer.

Alternatively, the method of constructing the magnetic array may involve magnetizing a single ferromagnetic substrate with the poles of the first layer and subsequently magnetizing the same ferromagnetic substrate with poles of the second layer.

In embodiments, the skin care active is niacinamide, that has a diamagnetic susceptibility score of −66 [10⁻⁶ cm³/mol]. Alternatively, or additionally, the skin care active may be a peptide, such as Pal Kttks or inositol.

According to a second aspect of the invention, there is provided a cosmetic skin care product comprising an applicator comprising a) a substrate with a magnetic array printed thereon, said magnetic array having at least one layer of one or more dipolar pairs of alternating magnetic poles; and b) a vibration source, and a skin care composition. The skin care composition may include one or more skin care actives with diamagnetic properties, for example, the skin care active may include one or more of a vitamin B3 active (e.g., niacinamide), a peptide (e.g., Pal KTTKS) or a sugar alcohol (e.g., inositol).

### BRIEF DESCRIPTION OF THE DRAWINGS

Embodiments of the invention will hereinafter be described, by way of example, with reference to the accompanying drawings, in which:

FIGS. 1A to 1C are perspective views of applicators of the skin care product described herein;

FIG. 2 shows an exploded view of the applicator of FIG. 1A;

FIG. 3A shows schematically a conventional bar magnet having a north and south pole;

FIG. 3B shows schematically a dipolar pair of magnets;

FIGS. 3C and 3D show schematically different arrangements of dipolar pairs in a magnetic array of the skin care product described herein;

FIGS. 4A to 4F illustrate schematically the magnetization and corresponding magnetic field generated in a magnetic array of the skin care product described herein;

FIGS. 5A and 5B illustrate schematically different ways of constructing a bi-directional magnetic array of the skin care product described herein;

FIG. 5C shows schematically a representation of the magnetic field generated by a bi-directional array;

FIG. 6 shows a bar chart illustrating penetration of inositol using an applicator of the present invention.

### DETAILED DESCRIPTION OF THE INVENTION

The skin care products disclosed herein exploit the benefits of enhanced penetration of skin care actives into skin via their unique diamagnetic properties, together with the mechanical benefits of using a vibrating applicator. Use of vibration in an applicator that is brought into contact with a keratinous surface, such as skin, is known to enhance penetration of skin care actives applied with the applicator or shortly before/after use of the applicator, through physical displacement of skin. Diamagnetism is the property of an object or material which causes it to create a magnetic field in opposition to an externally applied magnetic field, thus causing a repulsive effect. Surprisingly, it has been discovered that by combining vibration with a dipolar magnetic array in a skin care applicator, it is possible to further enhance penetration of actives into skin. Utilizing this discovery, it is possible to provide a cosmetic skin care product in which one or more skin care actives are delivered into skin to the point where they can provide a better skin care benefit than conventional skin care products. Without being bound by theory, it is thought that the diamagnetic force between the magnetic array in the applicator and the diamagnetic particles is strongest at the peaks of the magnetic flux generated by the dipolar magnets. By rapidly moving the applicator (through the vibratory movement), a greater number of diamagnetic particles “see” the peak magnetic flux in a shorter period of time (vs manual movement of a magnetic applicator over skin).

The cosmetic products disclosed herein provide enhanced penetration of skin care actives into skin. Methods of using the present skin products involve the use of a topical skin care composition in conjunction with an applicator that includes a vibration source and magnetic array purposefully designed to enhance penetration of skin care actives.

### Definitions

“Apply” or “application”, as used in reference to a composition, means to apply or spread the composition onto a surface of keratinous tissue.

“Derivative” refers to a molecule similar to that of another one, but differing from it in respect of a certain functional moiety.

“Disposed” refers to an element being located in a particular place or position relative to another element.

“Joined” means configurations whereby an element is directly secured to another element by affixing the element directly to the other element, and configurations whereby an element is indirectly secured to another element by affixing the element to intermediate member(s) that in turn are affixed to the other element.

“Keratinous tissue” refers to keratin-containing layers disposed as the outermost protective covering of mammals which includes, but is not limited to, skin, hair, nails, cuticles, etc.

“Magnetic field” and “magnetic flux density” are used interchangeably herein and refer to the vector field measured in teslas.

“Magnetic material” means a material that can be made into a permanent magnet.

“Pole” refers to the portion of a magnet that exhibits a higher magnetic flux density than the adjacent regions of the magnet. For example, a conventional bar magnet has two poles disposed at opposite ends where the magnetic flux density is highest.

“Regulating skin condition” means improving skin appearance and/or feel, for example, by providing a benefit, such as a smoother appearance and/or feel. Herein, “improving skin condition” means effecting a visually and/or tactilely perceptible positive change in skin appearance and feel. The benefit may be a chronic or acute benefit and may include one or more of the following: reducing the appearance of wrinkles and coarse deep lines, fine lines, crevices, bumps, and large pores; thickening of keratinous tissue (e.g., building the epidermis and/or dermis and/or sub-dermal layers of the skin, and where applicable the keratinous layers of the nail and hair shaft, to reduce skin, hair, or nail atrophy); increasing the convolution of the dermal-epidermal border (also known as the rete ridges), preventing loss of skin or hair elasticity, for example, due to loss, damage and/or inactivation of function skin elastin, resulting in such conditions as elastosis, sagging, loss of skin or hair recoil from deformation; reduction in cellulite; change in coloration to the skin, hair, or nails, for example, under-eye circles, blotchiness (e.g., uneven red coloration due to, for example, rosacea), sallowness, discoloration caused by hyperpigmentation, etc.

“Safe and effective amount” means an amount of a compound or composition sufficient to significantly induce a positive benefit, preferably a positive skin or feel benefit, including independently or in combinations the benefits disclosed herein, but low enough to avoid serious side effects (i.e., to provide a reasonable benefit to risk ratio), within the scope of sound judgment of the skilled artisan).

“Signs of skin aging” include, but are not limited to, all outward visibly and tactilely perceptible manifestations, as well as any macro- or micro-effects, due to keratinous tissue agine. These signs may result from processes which include, but are not limited to, the development of textural discontinuities such as wrinkles and coarse deep wrinkles, fine lines, skin lines, crevices, bumps, large pores, unevenness or roughness; loss of skin elasticity; discoloration (including under-eye circles); blotchiness; sallowness; hyperpigmented skin regions such as age spots and freckles; keratoses; abnormal differentiation; hyperkeratinization; elastosis; collagen breakdown, and other histological changes in the stratum corneum, dermis, epidermis, vascular system (e.g. telangiectasia or spider vessels), and underlying tissues (e.g., fat and/or muscle), especially those proximate to the skin.

“Skin” means the outermost protective covering of mammals that is composed of cells such as keratinocytes, fibroblasts and melanocytes. Skin includes an outer epidermal layer and an underlying dermal layer. Skin may also include hair and nails as well as other types of cells commonly associated with skin, such as, for example, myocytes, Merkel cells, Langerhans cells, macrophages, stem cells, sebocytes, nerve cells and adipocytes.

“Skin care” means regulating and/or improving a skin condition. Some nonlimiting examples include improving skin appearance and/or feel by providing a smoother, more even appearance and/or feel; increasing the thickness of one or more layers of the skin; improving the elasticity or resiliency of the skin; improving the firmness of the skin; and reducing the oily, skiny, and/or dull appearance of skin, improving the hydration status or moisturisation of the skin, improving the appearance of fine lines and/or wrinkles, improving skin exfoliation or desquamation, plumping the skin, improving skin barrier properties, improve skin tone, reducing the appearance of redness or skin blotches, and/or improving the brightness, radiancy, or translucency of skin.

“Skin care active” means a compound of combination of compounds that, when applied to skin, provide an acute and/or chronic benefit to skin or a type of cell commonly found therein. Skin care actives may regulate and/or improve skin or its associated cells (e.g., improve skin elasticity; improve skin hydration; improve skin condition; and improve cell metabolism).

“Skin care composition” means a composition that includes a skin care active and regulates and/or improves skin condition.

### Applicator

The cosmetic skin care product described herein includes a suitable applicator for either applying a skin care composition to a target portion of skin or placing above a target portion of skin to which a skin care composition has already been applied. The specific form of applicator may vary according to the intended target area of application on skin. For example, in some cases the skin care composition may be a whole body cream, and the applicator may be used to apply the cream to large surface body parts, for example, the legs, arms, abdomen and/or back. In this case, the magnetic array will need to be of a suitable size and shape to enable a user to quickly and easily cover a relatively large surface area. Alternatively, the skin care composition may be intended for use in smaller areas such as the face (e.g., cheeks, forehead, chin, nose, and peri-orbital regions). In such cases, the applicator and the magnetic array, which is discussed in more detail below, will be correspondingly shaped and sized.

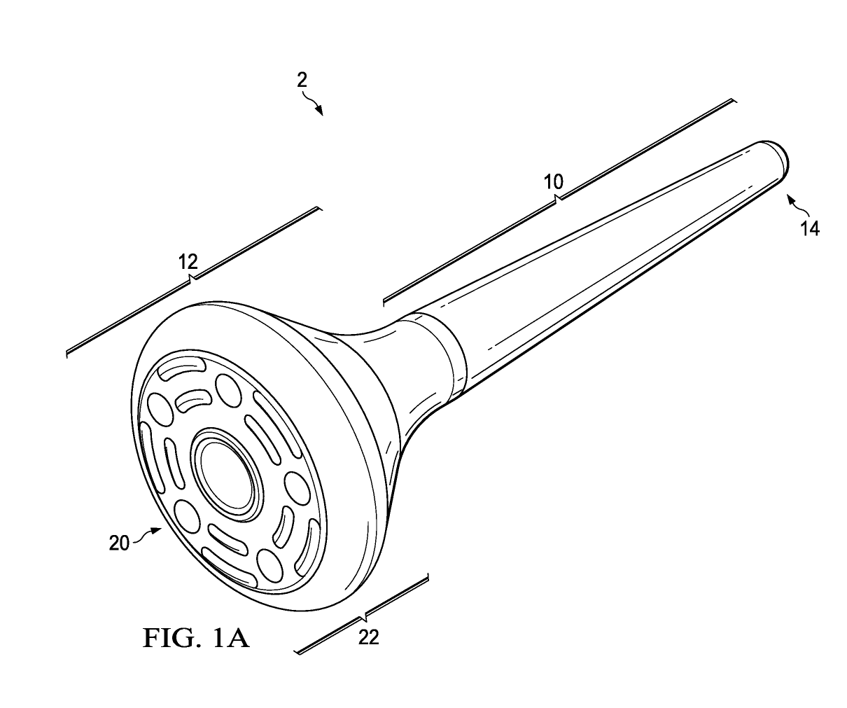

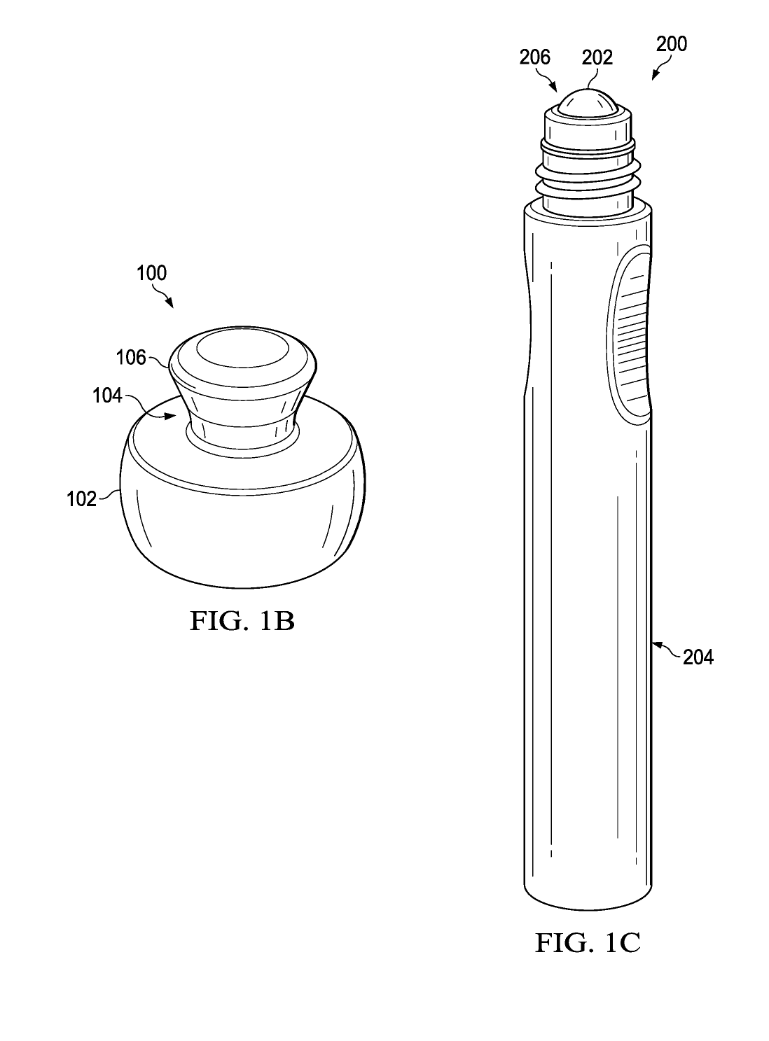

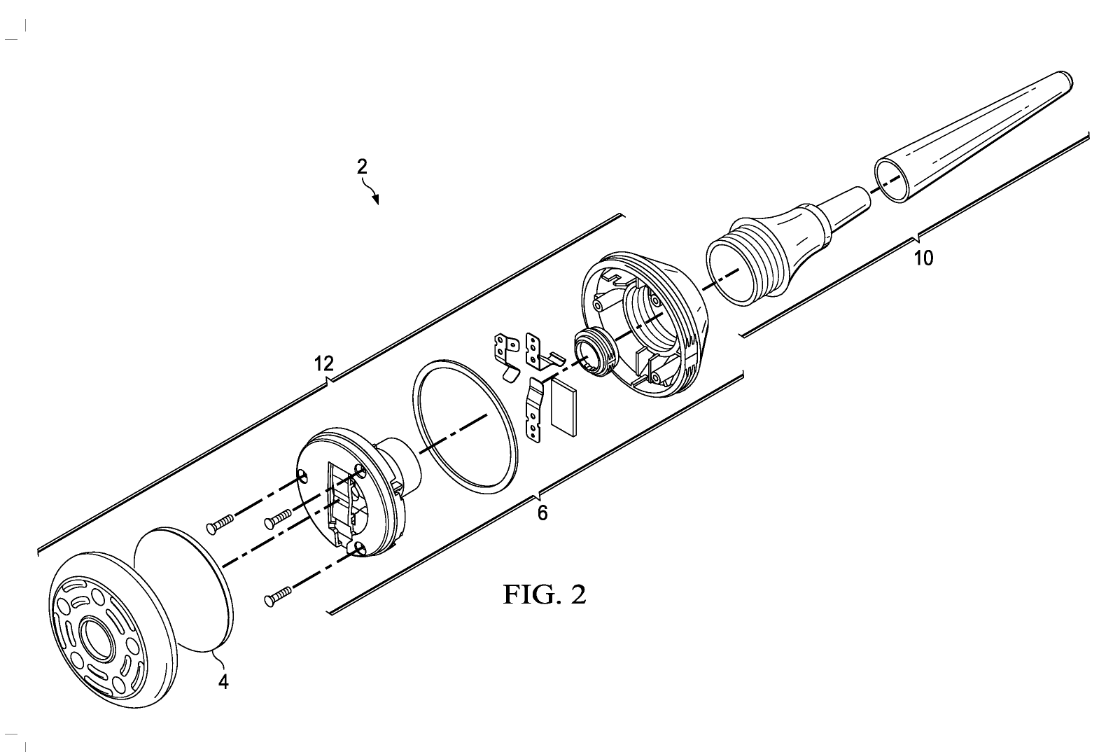

FIGS. 1A, 1B and 1C show examples of applicators 2, 100, 200 of the present invention. FIG. 2 is an exploded view of the applicator 2 shown in FIG. 1A, showing a magnetic substrate 4 upon which a magnetic array is imprinted and vibration source 6. The specific form of magnetic substrate/array and vibration source will be described in more detail below. However, in general, to be effective, the magnetic substrate should be positioned adjacent to and in parallel to a skin contact surface of the applicator.

The applicator 2 has a handle portion 10 and an applicator portion 12. The handle may be formed integrally with and of the same material as the cover. Alternatively, the handle may be formed of a different material to the cover, for example, the plastic, polymeric material or ceramic. In an example, the cover may be formed of polyvinyl chloride or rubber to provide a nice tactile handle for use during application of the skin care composition.

The handle portion may take any shape or size suitable for use as a cosmetic or skin care product applicator. In the embodiment shown in FIG. 1A, the applicator has an elongate tapered housing that is wider at the applicator portion 102 and gradually extends away to an apex 14. The housing that may easily be held between the finger and thumb of a user and enables the user to exert control over movement of the applicator across the surface of skin. Alternatively, as shown in FIG. 1(b), the handle of the applicator has a concave profile with a wide applicator portion 102, a narrow portion 104 of the handle proximate to the applicator tip and a wider portion 106 distal from the applicator tip. In this embodiment, the user is able to wrap their hand around the central, narrow portion of the handle, to exert control over movement of the applicator across the surface of skin.

The applicator 2 has a substantially annular tip located at the base of the handle intended for contact with a skin surface. The size and geometry of the tip will in general be determined by the intended application. For example, where the applicator is intended for use across the whole face or other larger body parts, a larger tip is provided, whereas for use around the eye, a smaller rounded tip may be useful. FIG. 1C shows an embodiment with rounded tip for use around the eye. The rounded tip 202 may be integrally formed with a handle 204, or it may be formed as a ball held within a socket 206 at the end of the handle 204. A magnetic array (not shown), formed of a flexible substrate is disposed inside the rounded tip 202, such that as the tip 202 is rolled over a surface of skin, the magnetic array will be substantially parallel to the surface of skin. Thus, the tip 202 functions as a cover for magnetic array disposed within the tip 202.

The applicator tip may be permanently joined to the applicator, or the tip may be removable, detachable and/or replaceable. It may be desirable for the tip to have a coefficient of friction that is less than that of the magnetic substrate of the magnetic array, which can provide a more desirable user experience when applying a skin care composition with the applicator. For example, the tip may have a dry coefficient of friction (i.e., a coefficient of friction measured without using a composition) that is between 10 and 50% less than the magnetic substrate (e.g., 15%, 20%, 25%, 30%, 35%, 40%, or even 45% less) according to the Friction Test described in the example below. When used to apply a skin care composition, the tip may exhibit a coefficient of friction that is up to 10 times less than the magnetic array (e.g., between 2× and 10× less, 3× and 7× or even between 4× and 6× less).

The tip may be formed from a material that provides a skin contacting surface with cooling properties. For example, the tip may be formed of a material that has a high thermal conductivity, for example, at least 50 W/mK, 100 W/mK or 200 W/mK. Providing a tip with high thermal conductivity cools the surface of skin to which it is applied. Because the thickness of the tip affects the distance that the magnetic flux density of the magnetic array extends, especially when formed from a non-magnetic material, it is important to ensure that the thickness of the cover does not undesirably inhibit the strength of the applied magnetic field. Suitable cover thicknesses are between 0.1 mm and 5 mm (e.g., between 0.2 and 4 mm, 0.5 and 3 mm, or even between 1 and 2 mm), for non-magnetic materials.

The applicator further comprises a power source for activation of the vibration source. In embodiments, the vibration source may be activated by a simple on/off switch. Alternatively, the vibration may be activated by one or more sensors, for example, proximity sensors that initiate the vibration upon contact with skin or pressure sensors that initiate the vibration upon increased pressure in the handle portion by a user. In embodiments, power for the vibration is activated upon completion of a circuit. In this embodiment, the applicator tip is formed of electrically conductive metal, and the base of the handle is formed of electrically conductive metal. If a user holds the applicator at the base of the handle on the metal area, the vibration source will be activated when the metal applicator tip is brought into contact with another body part.

### Magnetic Array

The present applicator includes a magnetic array specifically tailored to provide improved penetration of a specific skin care active, such as a vitamin B3 compound. The magnetic array described herein uses selectively magnetized permanent magnets (i.e., materials that create their own persistent magnetic field without an extrinsic power source such as a battery) to generate a magnetic field. The magnets may be formed of any one of numerous known ferromagnetic substrates, including, but not limited to: an iron compound (e.g., a ferrite such as barium ferrite, magnetite, or mild steel), a cobalt material, a strontium material, a barium material, a nickel material, alloys and oxides of these, combinations thereof and the like. The material may have a metalloid component such as boron, carbon, silicon, phosphorous or aluminium. Rare earth material such as neodymium or samarium may also be used.

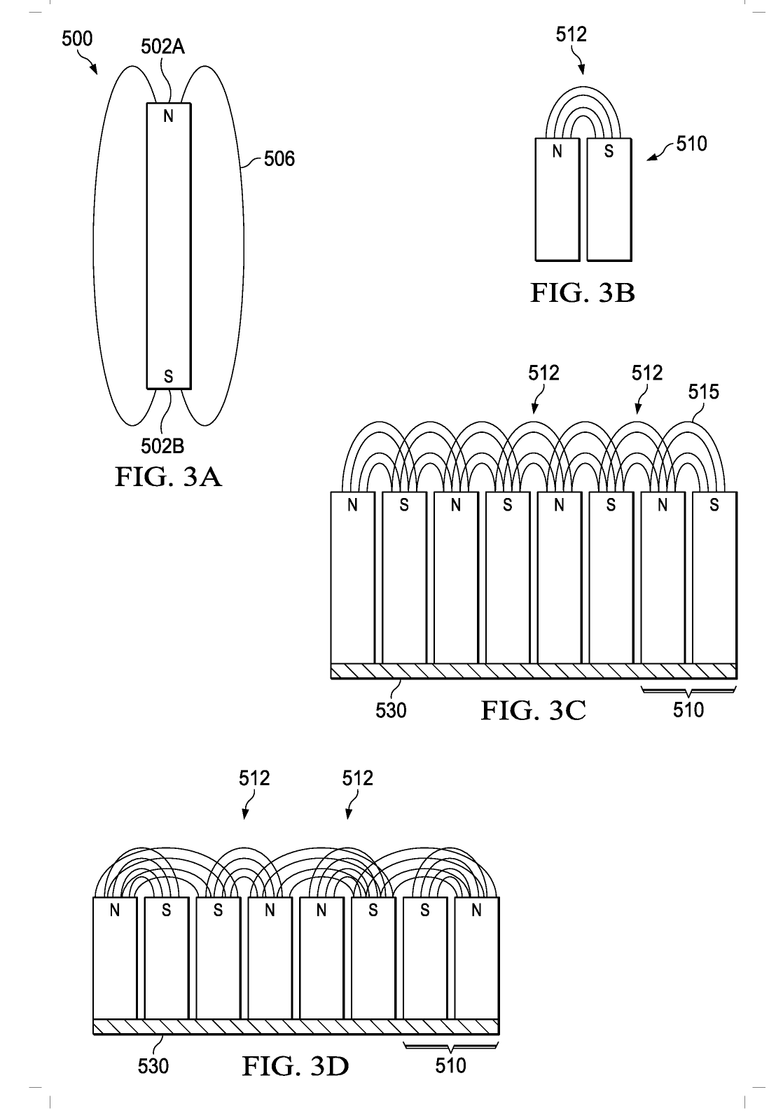

In a conventional bar magnet 500, the magnetic field 506 extends between opposite ends 502A and 502B, as shown in FIG. 3A. In contrast with a conventional bar magnet, the magnetic array(s) described herein are formed of one or more dipole pairs 510 of magnetic elements where magnetic poles of opposite polarity (N and S) are positioned adjacent one another and the magnetic field 512 extends between adjacent opposing poles, as illustrated schematically in FIG. 3B. For purposes of visualization, a dipole pair 510 may be thought of as a conventional rod magnet that is cleaved at its centre and the resulting sections brought together in a north-south (NS), side-by-side configuration.

The magnetic arrays herein may include multiple dipole pairs 510 arranged in series, and each dipole pair 510 can be in the same or a different orientation as that of the neighbouring pair (e.g., [NS][NS][NS] or [NS][SN][NS] as illustrated schematically in FIGS. 3C and 3D, respectively). Each dipole pair 510 generates its own magnetic field 512 that, in use, will induce a magnetic field in a diamagnetic material. The induced magnetic field of a diamagnetic material interacts repulsively with the applied magnetic field 512 of the dipole pairs 510 regardless of the direction of the applied field 512 (i.e., north or south). The magnitude of the repulsive force between the dipole pairs 510 and the diamagnetic material is determined by the magnetic flux density of the dipole pair 510 and the diamagnetic susceptibility of the diamagnetic material, in this case the skin care active. Magnetic flux density is generally greatest at the mid-point 515 between adjacent poles, and thus the strength of the magnetic field 512 will typically vary across the magnetic array depending on how the array is configured.

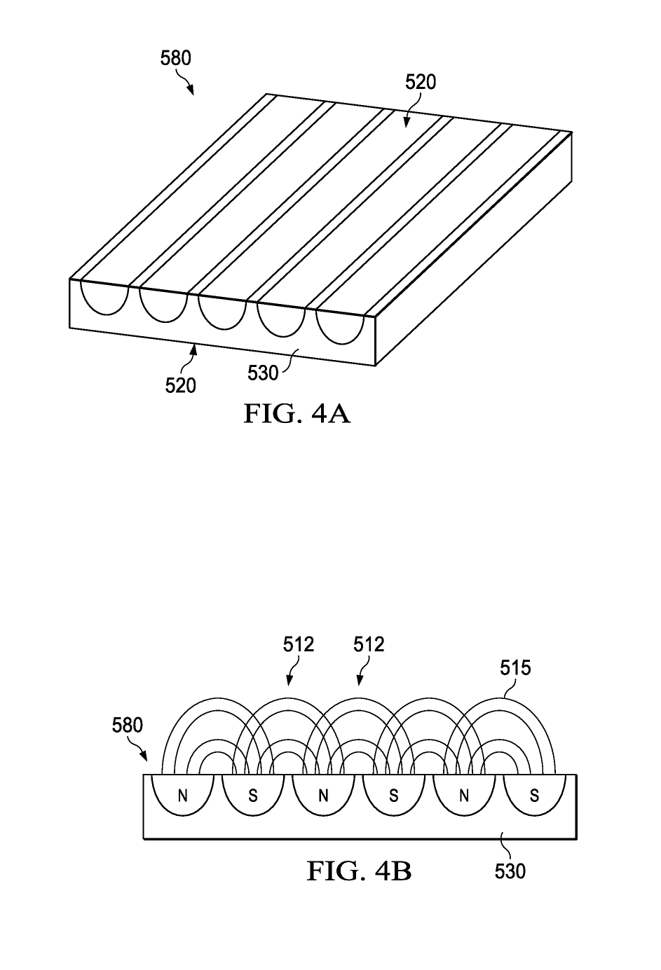

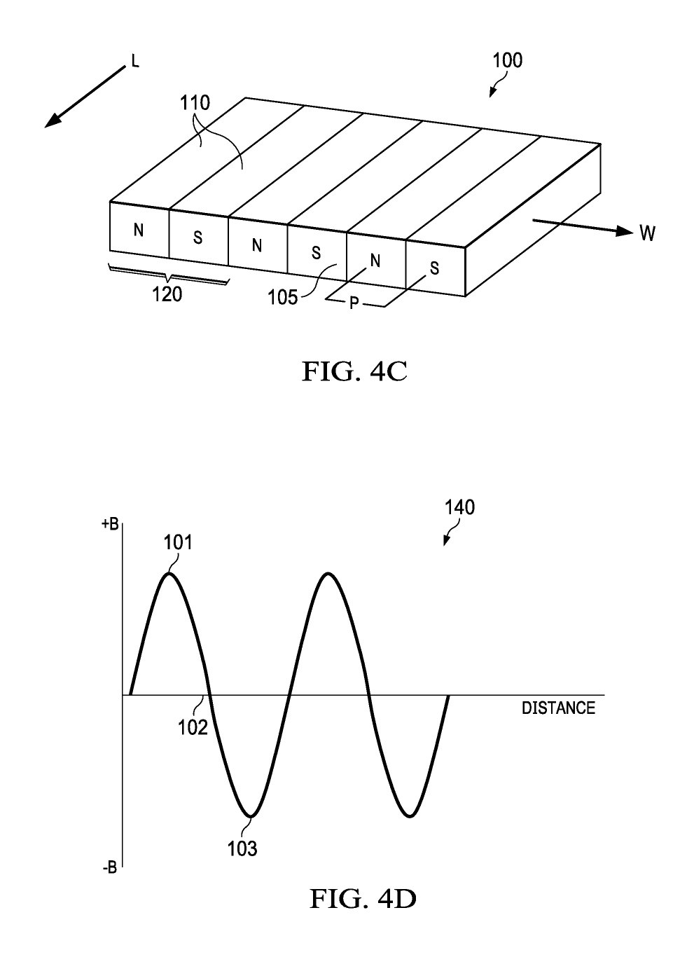

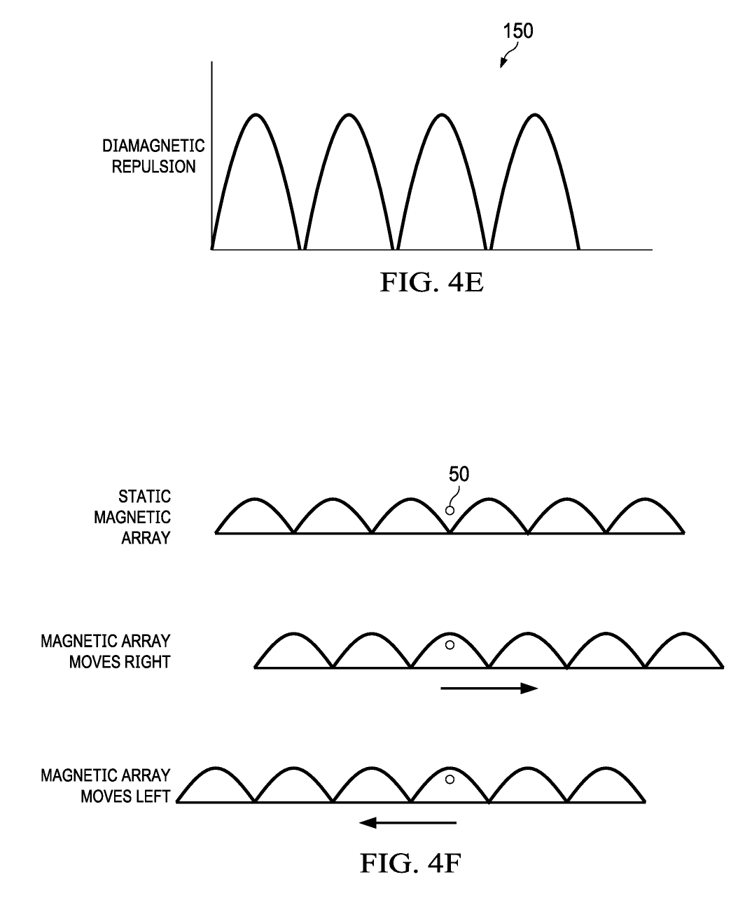

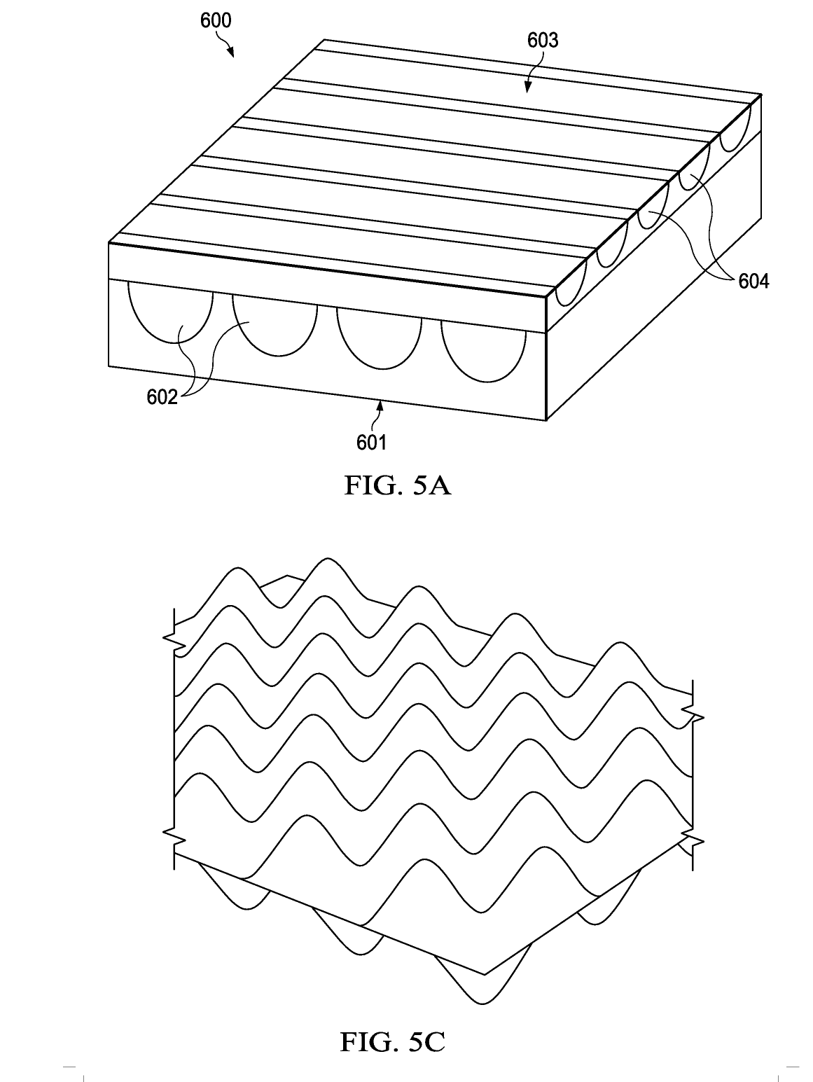

In practice, the substrate 580 used to form a magnetic array for use herein is typically not magnetized evenly throughout. As shown schematically in FIG. 4A, each pole extends from a first skin facing side 520 of the substrate 580 towards a second opposed side 522. An unmagnetized area 530 of substrate 580 is provided between each adjacent pole and at the second side 522 of the substrate 580. The unmagnetized area 530 at the second side 522 of the substrate 580 is known as the magnetic return. The magnetic return 530 is used to integrate the magnetic fields 512 generated by each pole on that side of the substrate and reduces or eliminates the magnetic field on that surface, instead diverting it towards the skin facing side 520. The magnetic return is preferably located on the side of the substrate 580 distal to the target biological surface to which the magnetic array is to be applied. The resultant magnetic field 512 is illustrated in FIG. 3B, where it can be seen that the magnetic field 512 extends outward from the first side 520 of the substrate 580, in a direction substantially perpendicular to the surface of the substrate 580, and is strongest at the mid-point 515 between adjacent opposing poles.

The magnetic array may be formed as a uni-directional array or a multi-directional array. In a uni-directional array, north (N) and south (S) poles are aligned in parallel to one another in a single layer, as shown in FIG. 4C. Adjacent poles are separated from one another by a pole centre-to-centre distance P, which defines the pitch of the magnetic array. FIG. 4C illustrates a portion of the magnetic field generated by the magnetic array in direction W that is perpendicular to the alignment of the poles. The waveform 140 illustrated in FIG. 4D shows the magnitude of the magnetic field varying regularly between +B and —B in a sinusoidal pattern, which corresponds to the difference in polarity (i.e., direction) of the magnetic field. The peaks and troughs of the waveform 140 correspond to the mid-point between adjacent poles. In other words, a first maximum magnetic flux density 101 occurs at the mid-point between a north to south pole, a minimum magnetic flux density 102 occurs in the centre of a pole, and a second maximum magnetic flux density 103 occurs at the mid-point between the adjacent south and north pole.

The amplitude of the waveform 140 is determined by the choice of magnetic substrate, the thickness or depth of substrate that is magnetized and the distance from the center of a pole to the edge of the pole. As the depth of magnetized area of a given substrate material increases, the maximum amplitude of the waveform 140 increases.

The frequency of the waveform 140 is determined by the pitch of the array. A higher pitch (i.e., greater center-to-center distance P) means that there are fewer magnetic flux density “maximums” per area of substrate, and thus a lower overall magnetic field strength for the array. However, a lower pitch may result in respective poles being packed too closely to one another for any single pole to reach its maximum potential magnetic flux density.

FIG. 4E illustrates the repulsive force that would be experienced by a diamagnetic material exposed to the magnetic field in FIG. 4D. As shown, the induced magnetic field of a diamagnetic material is independent of the direction of the magnetic field, and thus the change in the magnitude of the repulsive force corresponds to the change in magnitude of the applied magnetic field. FIG. 4F shows the impact on the diamagnetic molecule 50 as the applicator moved back and forth upon activation of the vibration source. Specifically, it can be seen that as the applicator vibrates, the diamagnetic material will experience the maximum magnetic field strength repeatedly, thus ensuring maximum and efficient penetration of the diamagnetic material into skin.

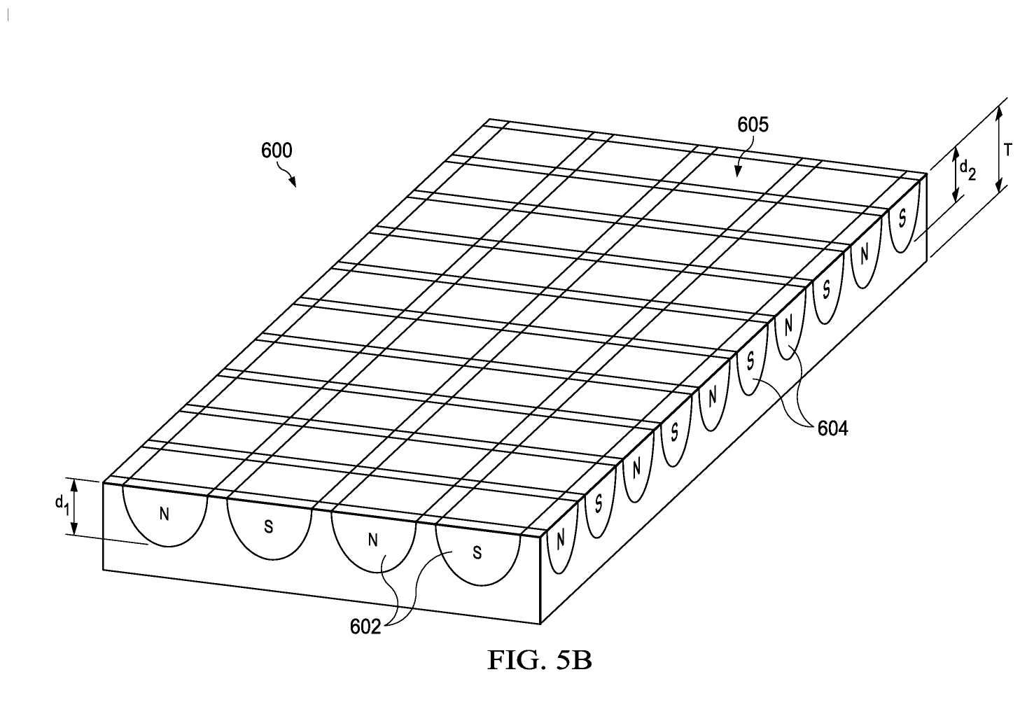

In some instances, the magnetic array may be formed as a multi-directional array, e.g., a bi-directional array, where multiple layers of parallel poles are juxtaposed at an angle relative to one another to provide multiple magnetic fields that constructively or destructively interfere with one another. In a multi-directional array, the magnetic flux density at any one point in the magnetic array will be determined by the combined magnetic flux density of poles of the different layers at that point. In some cases, this will lead to constructive interference where the resultant magnetic flux density at a point is greater than the magnetic flux density at that point for each individual layer, in other cases, the combination may lead to destructive interference where the resultant magnetic flux density at a point is less (sometimes zero) than the magnetic flux density at that point for each individual layer. FIG. 5C illustrates an example of the resultant magnetic field strength, shown in three dimensions of a bi-directional magnetic array. Since the induced magnetic field of a diamagnetic material is independent of the direction of the magnetic field, all areas of positive and negative magnetic field strength will appear as a repulsive force to a diamagnetic material.

Two layers of poles are provided in a bi-directional array, with both layers playing different roles. For example, the first layer of poles may determine the maximum magnetic field strength, while the second set of poles smoothes out the overall profile of the magnetic field, which reduces the instances of minimum magnetic flux density and ineffectual magnetic field strength. In some instances, use of such a bi-directional array makes it possible to better control the manner in which diamagnetic materials are pushed away from the magnetic array.

In practice, there are a variety of ways to form a bi-directional array 600. For example, as illustrated in FIG. 5A, the first 602 and second 604 layers of poles may be formed in two separate magnetic substrates 601 and 603, respectively, that are subsequently juxtaposed at an angle offset from one another. The magnetic return of both substrates 601 and 603 is positioned to face in the same direction such that the magnetic field generated by both layers of poles 602 and 604 extends away from the combined array in the same direction. The layers of poles 602 and 604 may be identical to one another (for example, having the same pitch between adjacent poles and the same maximum field strength), or the two layers 602 and 604 may vary in their specific parameters. Where the parameters of the two layers 602 and 604 vary, it is preferable for the layer that is proximal the target diamagnetic material (in FIG. 5A, the second layer 604) to be formed of a thinner substrate than that of the distal layer (in FIG. 5A, the first layer 602), otherwise the induced magnetic field of the diamagnetic material will be primarily based on the magnetic field strength of the proximal layer 604.

FIG. 5B illustrates an example in which the first 602 and second 604 layers of poles are formed in the same magnetic substrate 605. As shown in FIG. 5B, the substrate 605 is first magnetized in one direction to form a first layer 602 of parallel aligned north and south poles, and then remagnetized in a different direction with a second layer 604 of parallel aligned north and south poles to effectively form a woven pattern of poles. In this embodiment, the depth d2 of poles in the second layer 604 is equal to or less than the depth d1 of poles in the first layer 602. The depth d1 of the first layer 602 of poles is typically determined by the thickness T of the magnetic substrate 605.

The combined overall magnetic field strength of a magnetic array is measured after completion of the magnetization process using any known Gaussmeter. For bi-directional magnetic arrays made of two separate substrates, the overall magnetic field strength can be measured first for the respective layers and subsequently for the combined bi-directional magnetic array. In a bi-directional magnetic array, the overall magnetic field strength will approximately equate to the sum of the field strength of the individual layers.

Dipolar pairs of the magnetic substrate may be separated from adjacent dipolar pairs by a magnetically insulating material (i.e. a material with a relatively low magnetic permeability). In some instances, the magnetic elements may be arranged as individual segments or sections of magnetized ferromagnetic materials. Additionally or alternatively, the magnetic elements may be disposed in or on a solid or semi-solid substrate in which the required magnetic pattern is impressed upon the ferromagnetic particles or elements. The magnetic elements may be rigid elements within the applicator itself or disposed on a suitable substrate and joined to the applicator, for example, with an adhesive. In some instances, it may be desirable to embed the magnetic elements in a flexible matrix such as rubber or silicone and join the resultant array to a skin facing surface of the applicator.

When pairing a magnetic array with a skin care active such as a vitamin B3 compound, it is important for the magnetic field of the array to be tuned to interact with the diamagnetic susceptibility of the subject skin care active(s). If the magnetic field is improperly configured, for example, if the magnetic flux density is too low or the pitch between adjacent poles too great, there may be little to no magnetic field induced in the diamagnetic materials. Alternatively, if the magnetic flux density is too high, it may induce thermal noise and other forms of molecular entropy or disorder that act against the magnetic enhanced penetration of the skin care active. In some instances, even small departures from the proper configuration of the magnetic array may result in unsatisfactory penetration of the skin care actives.

In a particularly suitable example of a skin care product, a magnetic array is paired with a skin care composition that includes niacinamide. Niacinamide has a diamagnetic susceptibility of approximately −66 [10⁻⁶ cm³/mol]. Magnetic arrays suitable for enhancing the penetration of niacinamide include uni-directional and/or bi-directional arrays that exhibit enhanced penetration of skin care actives with a diamagnetic susceptibility of between −50[10⁻⁶ cm³/mol] and −80[10⁻⁶ cm³/mol]. The substrate may be formed of strontium ferrite powder impregnated in a polyvinyl chloride PVC base. A suitable uni-directional array may have a thickness of between 0.2 mm, 0.3 mm, 0.4 mm or 0.5 mm and 0.6 mm, 0.7 mm, 0.8 mm, 0.9 mm or 1 mm, a pitch (center to center distance between poles) of 1 mm, 1.5 mm or 2 mm to 2.5 mm, 3 mm or 3.5 mm between adjacent poles, leading to an overall magnetic field strength of between 12 mT, 14 mT, 15 mT, 17.5 mT or 20 mT to 22.5 mT, 25 mT, 28 mT or 30 mT. In a particularly suitable example of a uni-directional magnetic array, the magnetic array has an overall magnetic field strength of approximately 23 mT, a thickness of 0.6 mm and a pitch of about 2.1 mm (e.g., 12 poles per 25.4 mm).

An example of a suitable bi-directional array for enhancing penetration of a vitamin B3 compound into skin may have a first layer thickness of between 0.2 mm, 0.3 mm, 0.4 mm or 0.5 mm and 0.6 mm, 0.7 mm, 0.8 mm, 0.9 mm or 1 mm, a first layer pitch (center to center distance between poles) of 1 mm, 1.5 mm or 2 mm to 2.5 mm, 3 mm or 3.5 mm between adjacent poles, leading to a first layer magnetic field strength of between 12 mT, 14 mT, 17.5 mT or 20 mT to 22.5 mT, 25 mT, 28 mT or 30 mT, and a second layer thickness of between 0.05 mm, 0.1 mm, 0.15 mm or 0.2 mm and 0.25 mm, 0.3 mm, 0.4 mm or 0.6 mm, a second layer pitch of 1 mm, 1.25 mm or 1.5 mm to 2.5 mm, 3 mm or 3.5 mm between adjacent poles, leading to a second layer magnetic field strength of between 8 mT, 10 mT, 12 mT, 14 mT or 16 mT and 18 mT, 20 mT, 22 mT or 24 mT. The overall magnetic field strength of the bi-directional array may be between 14 mT and 30 mT. Typically, in a bi-directional array, the second layer magnetic field strength will be less than or equal to the first layer magnetic field strength and/or second layer pitch will be equal to or less than the first layer pitch. The first and second layers of the bi-directional array in this example may be angularly offset by between 1, 30, 45, 60 or 90 degrees and 120, 140, 160 or 179 degrees.

In a particularly suitable example of a bi-directional array, the magnetic array has an overall magnetic field strength of 27 mT, a first layer thickness of 0.6 mm, a first layer pitch of 2.1 mm (12 poles per 25.4 mm) and a second layer thickness of 0.2 mm and second layer pitch of 1.49 mm (17 poles per 25.4 mm).

The first layer of a bi-directional array may be formed of a uni-directional array.

In a particularly suitable example of a skin care product, a magnetic array is paired with a skin care composition that includes Pal-KTTKS. Pal-KTTKS has a diamagnetic susceptibility of approximately −519. Magnetic arrays suitable for enhancing the penetration of Pal-KTTKS include uni-directional and/or bi-directional arrays that exhibit enhanced penetration of cosmetic actives with a diamagnetic susceptibility of between about −400 and −600. A suitable example of a uni-directional magnetic array for enhancing the penetration of Pal-KTTKS into skin is a magnetic array formed from a strontium ferrite powder impregnated in a polyvinyl chloride PVC base. In this example, the magnetic array may have a thickness of between 0.9 and 1.3 mm (e.g., 1.0, 1.1 or 1.2 mm); a pitch of between 1.7 and 2.5 mm (e.g., 1.8, 1.9, 2.0, 2.1, 2.2, 2.3 or 2.4 mm); and an overall field strength of from 24.0 to 36.0 mT (e.g., about 24.5, 25, 25.5, 26, 26.5, 27, 27.5, 28, 28.5, 29. 29.5, 30, 30.5, 31, 31.5, 32, 32.5, 33, 33.5, 34, 34.5, or even about 35 mT). In a particularly suitable example of a uni-directional magnetic array, the magnetic array has an overall magnetic field strength of approximately 27 mT, a thickness of 1.1 mm and a pitch of about 2.1 mm (e.g., 12 poles per 25.4 mm).

An example of a suitable bi-directional array for enhancing the penetration of Pal-KTTKS into skin may have a first layer thickness of between about 0.3 and 0.9 mm (e.g., 0.4, 0.5, 0.6, 0.7 or even 0.8 mm) and a first layer pitch of between 1.7 and 2.5 mm or about 12 poles per 25.4 mm (e.g., a pitch of 1.8, 1.9, 2.0, 2.1, 2.2, 2.3 or 2.4 mm), leading to a first layer magnetic field strength of between 20 mT and 26 mT (e.g., 21, 22, 23, 24 or even 25 mT), especially about 23.2 mT. The bi-directional array in this example may have second layer thickness of between 0.05 mm and 0.5 mm (e.g., 0.1, 0.15, 0.2, 0.25, 0.3. or even 0.4 mm) and a second layer pitch of about 0.8 mm to about 1.3 mm or 25 poles per 25.4 mm (e.g., a pitch of between 0.9 and 1.2 mm or between 1.0 and 1.1 mm), leading to a second layer field strength of between 1 mT and 24 mT (e.g., 2, 3, 4, 5, 6, 7, 8, 9, 10, 11, 12, 13, 14, 15, 16, 17, 18, 19, 20, 21, 22, or 23 mT). The overall magnetic field strength of the bi-directional array may be between 14 mT and 30 mT (e.g., 15, 16, 17, 18, 19, 20, 21, 22, 23, 24, 25, 26, 27, 28 or 29 mT). The bi-directional array may have an overall magnetic field strength of between about 19.0 and about 25.0 mT (e.g., 20, 21, 22, 23, or even 24 mT). Typically, in a bi-directional array, the magnetic field strength of the second layer will be less than or equal to the magnetic field strength of the first layer, and/or the second layer pitch will be less than or equal to the first layer pitch. The first and second layers of the bi-directional array in this example may be formed from uni-directional arrays that are angularly offset by between 1 and 179 degrees (e.g., between 45 and 135 degrees, between 60 and 120 degrees, or even about 90 degrees).

### Vibration Source

In embodiments, the vibration source may have an amplitude of vibration of up to 1 mm, 1.5 mm or 2 mm. The vibration source may have a minimum amplitude of 0.1 mm, 0.2 mm, 0.4 mm or 0.5 mm preferably, the vibration source has an amplitude of about half the pitch between adjacent peaks of the dipolar magnet. Where the amplitude of vibration is about half the pitch between adjacent peaks, in theory all diamagnetic particles should experience the maximum diamagnetic force at some point and, preferably, repeatedly.

The vibration source may generate vibration of frequency lying in the range of from 0.5, 1, 10 or 50 hertz (Hz) to 200, 300, 500 or 1000 hertz (Hz). Increasing the frequency helps to ensure that all diamagnetic particles receive the same amount of magnetic force from the magnetic array. However, if the frequency is too high, the feel on the skin for a user may be unpleasant.

The vibration may be produced intermittently or continuously.

Typically, the vibration source comprises a motor, e.g., a disk-shaped or pancake motor, driving an off-center fly-weight in rotation. The speed of rotation of the motor may lie in the range 2500 revolutions per minute (RPM), 4500 RPM or 6500 RPM to 7000 RPM, 10000 RPM, 12500 RPM or 15000 (RPM), preferably in the range 4500 RPM to 10000 RPM.

The use of a motor driving an off-center fly-weight in rotation makes it possible to generate vibration of amplitude that is relatively small and of frequency that is relatively high.

Alternatively and/or additionally, the vibration source may be piezoelectric, electromechanical or eccentric. The vibration source may comprise a motor driving, in rotation, a toothed wheel that is in contact with an elastically-deformable blade, like a rattle.

The voltage may lie in the range of 1.3 volts (V) to 9V, for example. The vibration source may include an electricity source, such as 1.5V button battery. The use of a button battery may be advantageous to provide a compact device. When using a button battery and a disk-shaped motor, the battery and the motor may be face-to-face, side-by-side, or the face of one may face the edge of the other.

The applicator further comprises a power source for activation of the vibration source. In embodiments, the vibration source may be activated by a simple on/off switch. Alternatively, the vibration may be activated by one or more sensors, for example, proximity sensors that initiate the vibration upon contact with skin or pressure sensors that initiate the vibration upon increased pressure in the handle portion by a user. In embodiments, power for the vibration is activated upon completion of a circuit. In this embodiment, the applicator tip is formed of electrically conductive metal, and the base of the handle is formed of electrically conductive metal. If a user holds the applicator at the base of the handle on the metal area, the vibration source will be activated when the metal applicator tip is brought into contact with another body part. In this respect, the circuit is completion when the tip touches the skin surface to which the product is to be applied, and the user grabs the metal portion at the base of the handle. The contacts are part of a touch switch circuit that activates a vibration motor upon detecting a current path.

Alternatively and/or additionally, the applicator preferably includes a control member for controlling operation of the vibration source. The control member may be a simple on/off switch or trigger, operated by the user. The device may include a control member for controlling operation of the vibration source. The control member may be triggered by pressing it. The control member may be disposed in such a manner that it is pressed when the user takes hold of the device. The control member may operate upon completion of a circuit where

The vibration produced may be oriented substantially parallel to a longitudinal axis of the applicator. In a variant, the vibration may be substantially perpendicular to the longitudinal axis of the applicator.

### Skin Care Composition

A skin care composition of the present invention may be applied to mammalian keratinous tissue, in particular to human skin. The cosmetic compositions may take various forms. For example, some non-limiting examples of forms include solutions, suspensions, lotions, creams, gels, toners, sticks, pencils, sprays, aerosols, ointments, cleansing liquid washes and solid bars, shampoos and hair conditioners, pastes, foams, powders, mousses, shaving creams, wipes, strips, patches, electrically-powered patches, wound dressing and adhesive bandages, hydrogels, film-forming products, facial and skin masks, cosmetics (e.g. foundations, eye liners, eye shadows), and the like.

Skin care compositions may include a first skin care active such as a vitamin B3 compound, for example niacin or niacinamide. As used herein, “vitamin B3 compound” means a compound having the formula:

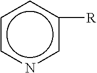

wherein R is —CONH₂ (i.e., niacinamide), —COOH (i.e., nicotinic acid) or —CH2OH (i.e., nicotinyl alcohol); derivatives thereof; and salts of any of the foregoing.

Suitable esters of nicotinic acid include nicotinic acid esters of C₁-C₂₂, preferably C₁-C₁₆, more preferably C₁-C₆ alcohols. The alcohols are suitably straight-chain or branched chain, cyclic or acyclic, saturated or unsaturated (including aromatic), and substituted or unsubstituted. The esters are preferably non-vasodilating. As used herein, “non-vasodilating” means that the ester does not commonly yield a visible flushing response after application to the skin in the subject compositions (the majority of the general population would not experience a visible flushing response, although such compounds may cause vasodilation not visible to the naked eye, i.e., the ester is non-rubifacient). Non-vasodilating esters of nicotinic acid include tocopherol nicotinate and inositol hexanicotinate; tocopherol nicotinate is preferred.

Other derivatives of the vitamin B₃ compound are derivatives of niacinamide resulting from substitution of one or more of the amide group hydrogens. Nonlimiting examples of derivatives of niacinamide useful herein include nicotinyl amino acids, derived, for example, from the reaction of an activated nicotinic acid compound (e.g., nicotinic acid azide or nicotinyl chloride) with an amino acid, and nicotinyl alcohol esters of organic carboxylic acids (e.g., C1-C18). Specific examples of such derivatives include nicotinuric acid (C8H8N2O3) and nicotinyl hydroxamic acid (C6H6N2O2), which have the following chemical structures:

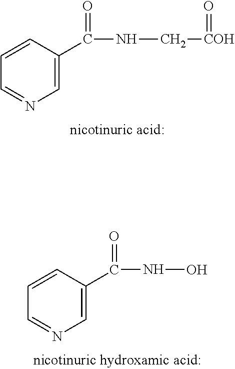

Exemplary nicotinyl alcohol esters include nicotinyl alcohol esters of the carboxylic acids salicylic acid, acetic acid, glycolic acid, palmitic acid and the like. Other non-limiting examples of vitamin B3 compounds useful herein are 2-chloronicotinamide, 6-aminonicotinamide, 6-methylnicotinamide, n-methyl-nicotinamide, n,n-diethylnicotinamide, n-(hydroxymethyl)-nicotinamide, quinolinic acid imide, nicotinanilide, n-benzylnicotinamide, n-ethylnicotinamide, nifenazone, nicotinaldehyde, isonicotinic acid, methyl isonicotinic acid, thionicotinamide, nialamide, 1-(3-pyridylmethyl) urea, 2-mercaptonicotinic acid, nicomol, and niaprazine.

Examples of the above vitamin B3 compounds are well known in the art and are commercially available from a number of sources, e.g., the Sigma Chemical Company (St. Louis, Mo.); ICN Biomedicals, Inc. (Irvin, Calif.) and Aldrich Chemical Company (Milwaukee, Wis.).

One or more vitamin B3 compounds may be used herein. Preferred vitamin B3 compounds are niacinamide and tocopherol nicotinate. Niacinamide is more preferred.

When used, salts, derivatives, and salt derivatives of niacinamide are preferably those having substantially the same efficacy as niacinamide.

Salts of the vitamin B3 compound are also useful herein. Nonlimiting examples of salts of the vitamin B3 compound useful herein include organic or inorganic salts, such as inorganic salts with anionic inorganic species (e.g., chloride, bromide, iodide, carbonate, preferably chloride), and organic carboxylic acid salts (including mono-, di- and tri-C1-C18 carboxylic acid salts, e.g., acetate, salicylate, glycolate, lactate, malate, citrate, preferably monocarboxylic acid salts such as acetate). These and other salts of the vitamin B3 compound can be readily prepared by the skilled artisan, for example, as described by W. Wenner, “The Reaction of L-Ascorbic and D-Iosascorbic Acid with Nicotinic Acid and Its Amide”, J. Organic Chemistry, Vol. 14, 22-26 (1949). Wenner describes the synthesis of the ascorbic acid salt of niacinamide.

In a preferred embodiment, the ring nitrogen of the vitamin B3 compound is substantially chemically free (e.g., unbound and/or unhindered), or after delivery to the skin becomes substantially chemically free (“chemically free” is hereinafter alternatively referred to as “uncomplexed”). More preferably, the vitamin B3 compound is essentially uncomplexed. Therefore, if the composition contains the vitamin B3 compound in a salt or otherwise complexed form, such complex is preferably substantially reversible, more preferably essentially reversible, upon delivery of the composition to the skin. For example, such complex should be substantially reversible at a pH of from about 5.0 to about 6.0. Such reversibility can be readily determined by one having ordinary skill in the art.

More preferably the vitamin B3 compound is substantially uncomplexed in the composition prior to delivery to the keratinous tissue. Exemplary approaches to minimizing or preventing the formation of undesirable complexes include omission of materials which form substantially irreversible or other complexes with the vitamin B3 compound, pH adjustment, ionic strength adjustment, the use of surfactants, and formulating wherein the vitamin B3 compound and materials which complex therewith are in different phases. Such approaches are well within the level of ordinary skill in the art.

Thus, in a preferred embodiment, the vitamin B3 compound contains a limited amount of the salt form and is more preferably substantially free of salts of a vitamin B3 compound. Preferably the vitamin B3 compound contains less than about 50% of such salt, and is more preferably essentially free of the salt form. The vitamin B3 compound in the compositions hereof having a pH of from about 4 to about 7 typically contain less than about 50% of the salt form.

The vitamin B3 compound may be included as the substantially pure material, or as an extract obtained by suitable physical and/or chemical isolation from natural (e.g., plant) sources. The vitamin B3 compound is preferably substantially pure, more preferably essentially pure.

In some examples, the cosmetic compositions may have a concentration of a vitamin B3 compound, by weight of the cosmetic composition, of greater than 0.0005%, 0.00056%, 1%, 2%, 3%, 4%, or 5% and/or less than 11%, 10%, 8%, or 6%.

The topical application of niacinamide may be associated with a variety of cosmetic skin care benefits. These may include: i) normalization of age associated depletions of nicotinamide coenzymes in skin, ii) up-regulation of epidermal ceramide synthesis with concurrent epidermal barrier benefits, iii) protection against damage produced by UV irradiation, iv) inhibition of the transfer of melanosomes from melanocytes to keratinocytes (thereby providing a potential skin tone benefit), and reduction in sebaceous lipogenesis. Thus, in certain instances, it may be desirable to include niacinamide in the cosmetic composition in order to improve the appearance of aging/photo-damaged skin.

The cosmetic compositions may also comprise a dermatologically acceptable carrier (which may also be referred to as a “carrier”) within which the vitamin B3 compound is incorporated to enable the compound and optional other ingredients to be delivered to the skin. The carrier may contain one or more dermatologically acceptable solid, semi-solid or liquid fillers, diluents, solvents, extenders components, materials and the like. The carrier may be solid, semi-solid or liquid. The carrier may be provided in a wide variety of forms. Some non-limiting examples include simple solutions, (aqueous or oil based), emulsions, and solid forms (e.g., gels, sticks, flowable solids, amorphous materials).

The carriers may contain one or more dermatologically acceptable, hydrophilic diluents. Hydrophilic diluents include water, organic hydrophilic diluents such as lower monovalent alcohols (e.g., C1-C4) and low molecular weight glycols and polyols, including propylene glycol, polyethylene glycol (e.g., molecular weight 200-600 g/mole), polypropylene glycol (e.g., molecular weight 425-2025 g/mole), glycerol, butylene glycol, 1,2,4-butanetriol, sorbitol esters, 1,2,6-hexanetriol, ethanol, isopropanol, sorbitol esters, butanediol, ether propanol, ethoxylated ethers, propoxylated ethers and combinations thereof.

Carriers may also be in the form of an emulsion, such as oil-in-water emulsions, water-in-oil emulsions, and water-in-silicone emulsions. An emulsion may generally be classified as having a continuous aqueous phase (e.g., oil-in-water and water-in-oil-in-water) or a continuous oil phase (e.g., water-in-oil and oil-in-water-in-oil). The oil phase may comprise silicone oils, non-silicone oils such as hydrocarbon oils, esters, ethers, and the like, and mixtures thereof. The aqueous phase may comprise water, such as a solution as described above. However, in other embodiments, the aqueous phase may comprise components other than water, including but not limited to water-soluble moisturizing agents, conditioning agents, anti-microbials, humectants and/or other water-soluble skin care actives.

Various cosmetic treatments may be employed. Skin surfaces of the most concern tend to be those not typically covered by clothing such as facial skin surfaces, hand and arm skin surfaces, foot and leg skin surfaces, and neck and chest skin surfaces. In particular, facial skin surfaces, including the forehead, peri-oral, chin, peri-orbital, nose, and/or cheek skin surfaces, may be treated with the cosmetic compositions described herein.

In an alternative embodiment, the skin care composition comprises a safe and effective amount of Pal-KTTKS, for example, Matrixyl® or Promatrixyl® brand Pal-KTTKS (100 ppm Pal-KTTKS) available from Sederma, France. The Pal-KTTKS may be included in the present skin care composition at an amount of from 1×10-6% to 10% by weight of the composition (e.g., 1×10-6% to 0.1%, even from 1×10-5% to 0.01%). In embodiments wherein Matrixyl® or Promatrixyl® is used, the resulting composition preferably contains from 0.01% to 50%, by weight of the resulting composition, of Matrixyl® or Promatrixyl® (e.g., from 0.05% to 20%, or from 0.1% to 10%). The present skin care compositions may include additional optional ingredients known for safe use in skin care compositions (e.g., emollients, humectants, vitamins; peptides; and sugar amines, sunscreen actives (or sunscreen agents), ultraviolet light absorbers, colorants, surfactants, film-forming compositions, and rheology modifiers). Some non-limiting examples of optional ingredients for use in the present compositions are disclosed in U.S. Publication No. US2008/0206373, filed by Millikin, et al., on Feb. 28, 2008.

In another alternative embodiment, the skin care composition comprises inositol as the skin care active. Inositol (cyclohexane-1,2,3,4,5,6-hexol; C₆H₁₂O₆) is a sugar alcohol that plays an important role as the structural basis for a number of secondary messengers and signaling molecules and is an important component of phosphatidylinositol. Inositol is an active material in topical formulations for reducing the appearance of age spots and improving the appearance of skin tone.

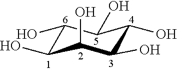

### Methods of Use

The skin care product described herein may be used for applying the skin care composition to one or more skin surfaces as part of a user's daily routine. A consumer may use the skin care product by dispensing a desired amount of the skin care composition onto the applicator and then, using the skin contact surface of applicator, applying the composition to a target area of the person's skin. In doing so, the magnetic array located within the applicator is able to act together with the diamagnetically susceptible material within the skin care composition to increase the volume of skin care active that penetrates into skin. The skin care composition may be applied to the applicator manually by the user (for example, by using the applicator to scoop some of the composition out of a tub) and/or the composition may be held in a reservoir provided in the applicator and dispensed automatically onto the skin contact surface of the applicator.

Additionally or alternatively, the skin care composition may be applied directly to a user's skin surface in a normal manner (i.e. by finger application) and the applicator subsequently swept over the target area of skin.

The skin care composition may be intended primarily for use on facial skin surfaces, including one or more of the cheek, forehead and peri-orbital areas of the face.

### EXAMPLES

The following examples are given solely for the purpose of illustration and are not to be construed as limiting the invention, as many variations thereof are possible.

### Example 1—Pal KTTKS In Vivo Skin Penetration Study

Pal KTTKS Enhancement from Cream (w/Vibration Vs. w/o Vibration)

| μg PalKTTKS/μg | | | | | | |
|---|---|---|---|---|---|---|
| skin of Strip 6-10 | Panelist1 | Panelist2 | Panelist3 | Panelist4 | Panelist5 | Panelist6 |
| Magnetic Micro array | 0.0107006 | 0.01869 | 0.00663 | 0.01973 | 0.05444 | 0.03195 |
| w/o Vibration | | | | | | |
| Magnetic Micro array | 0.0115249 | 0.02161 | 0.01304 | 0.0258 | 0.0568 | 0.04369 |
| w Vibration | | | | | | |
| Enhancement for | 1.0770281 | 1.156436 | 1.965565 | 1.307474 | 1.043383 | 1.36722 |
| strip 6-10 | | | | | | |

| μg PalKTTKS/μg | | | | | |
|---|---|---|---|---|---|
| skin of Strip 6-10 | Panelist7 | Panelist8 | Panelist9 | Panelist10 | Mean |
| Magnetic Micro | 0.0290154 | 0.01924 | 0.00638 | 0.01984 | 0.02166 |
| array w/o Vibration | | | | | |
| Magnetic Micro | 0.0218441 | 0.02081 | 0.01648 | 0.02061 | 0.02522 |
| array w Vibrations | | | | | s |
| Enhancement for | 0.75284624 | 1.081426 | 2.582488 | 1.038614 | 1.33725 |
| strip 6-10 | | | | | |

“s” indicates significant difference vs. mean of passive (p < 0.1)

An in vivo skin penetration study was conducted to establish the effect of using a skin care product of the present invention by applying a skin care composition comprising a peptide, in this case, Pal KTTKS, with an applicator comprising a magnetic array and a vibration source. The study used 10 active study sites (composition applied to target skin surface using an applicator containing a magnetic array and vibration source of the present invention) versus 10 control study sites (composition applied to target skin surface using an applicator with just a magnetic array). In this example, the level of Pal KTTKS present in the extract from each tape strip was measured using HPLC and the results normalized to the protein level measured on the tape strip. While Pal KTTKS is delivered into skin using the control applicator, it can clearly be seen that penetration of the active is enhanced when using an applicator combining the magnetic array with a vibration source.

### Tape Stripping Method

This method provides a suitable means of measuring the amount of skin care active present in skin, and comparing active versus passive application of the skin care active. Two identical circular areas of 7.9 cm² were marked on the volar forearms of volunteers. A measured dose (approximately 9 mg) of the skin care formulation (ingredients shown in Table 1) was applied to the delineated areas using a screw actuated syringe. Application was carried out in each case using purpose made applicators having a metallic aluminium skin contact surface behind which was the magnetic array. The cream was spread evenly across the entire delineated region using the applicator in a sweeping motion with a fixed speed of approximately 3.5 cm/s to mimic normal finger application. The application period was 30 seconds during which time visual inspection was used to ensure even distribution and absorption of the formulation by skin. The application area was then left uncovered for a further 30 minutes to ensure complete absorption. The application area was then wiped thoroughly to remove any surface material, followed by carrying out tape stripping.

The Tape Stripping method can be carried out using commercial pre-cut 22.1 mm tape stripping adhesive discs (D-Squame, Cuderm Corporation or equivalent) with an adhesive area of 3.8 cm². A 22.1 mm diameter circular region was marked at the centre of the application area. A tape stripping adhesive disc was placed over the marked area and even pressure applied using a neoprene roller, rolled ten times over the surface. The adhesive disc was removed from the skin surface in a single pulling motion using manual tweezers. To ensure even removal of the stratum corneum, subsequent discs were removed in a north, south, east and west orientation. Each adhesive disc was non-destructively analysed for relative protein content using the SquameScan™ 850 instrument (Heiland Electronics Wetzlar Germany). The adhesive disc was then immediately placed into a glass vial containing extraction solvent in preparation for subsequent analysis. Solvent extractions are conducted on each tape strip using conventional extraction methods, which are well known to those of ordinary skill in the art, and measuring the amount of niacinamide present in the extract, for example, by high performance liquid chromatography (“HPLC”) and/or mass spectrometry.

The procedure was repeated for the remaining nine strips. An additional strip was obtained from outside the area of application of the skin care formulation to serve as a blank sample.

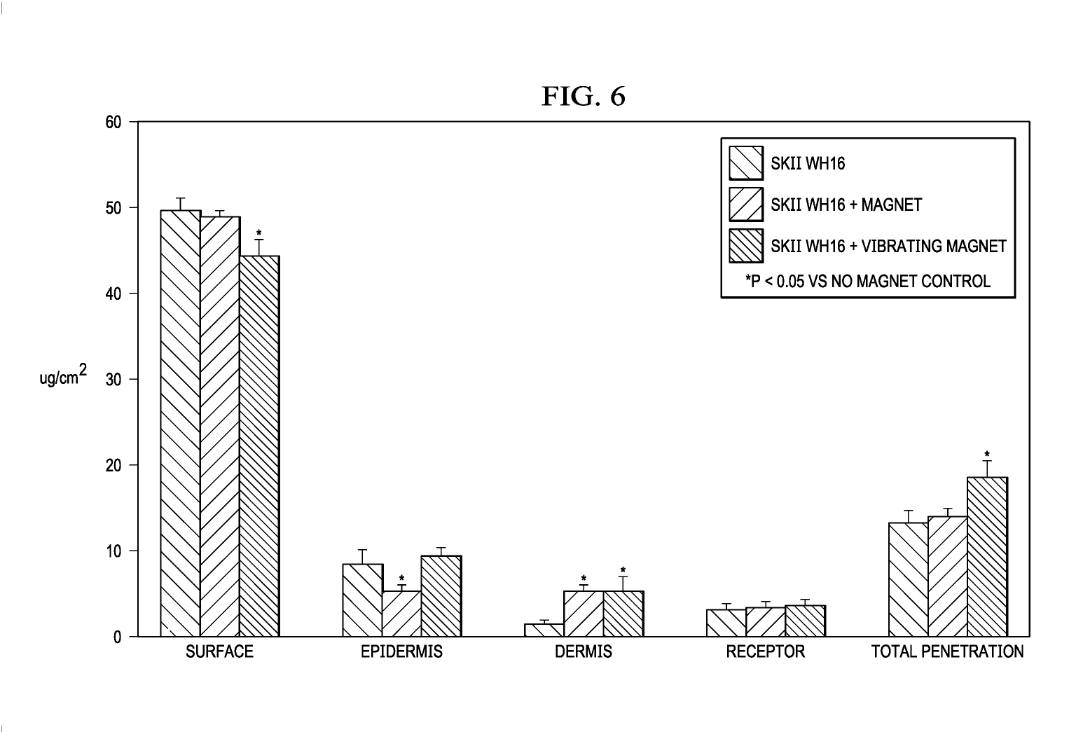

### Example 2—Inositol In Vitro Skin Penetration Study (See FIG. 6)

Skin penetration was measured using the Franz Diffusion Cell assay. Split-thickness (dermatomed) human cadaver skin was thawed under ambient conditions, cut into appropriately sized sections, and mounted in standard Franz-type diffusion cells (0.79 cm² surface area) maintained at about 37° C. The receptor compartments were filled with ˜5 mL phosphate buffered saline (PBS—pH 7.4) that included 1% polysorbate-20 and 0.02% sodium azide, and the skin was allowed to equilibrate for two hours. The cells were qualified upon ³H₂0 flux through the mounted skin (150 μL of ³H₂0 applied for five minutes and removed; receptor fluid was collected after one hour and analyzed using scintillation spectrometry). Diffusion cells were randomized by ranking each cell according to water flux and distributing cells across treatment legs such that each group included cells across the range of observed water flux.

Where indicated, aliquots of the test products/formulations were spiked with the appropriate radioactive material (³H-inositol, ¹⁴C-niacinamide, ¹⁴C-pal-KTTKS or ¹⁴C-retinyl propionate) with approximately 3 μCi per 300 mg product aliquot, mixed and assayed for total radioactivity in triplicate using Ultima Gold [Perkin-Elmer] liquid scintillation cocktail (LSC) and liquid scintillation counting.

Skin was topically dosed with ˜2.5 μL of product (containing 0.5% inositol in a standard skin care composition) using a positive displacement pipette. The product was gently spread over the surface of the skin (0.79 cm²) using the OBJ magnet with or without vibration or a curved end of a stainless steel spatula (sham control). At 24 hours post-application, the receptor solution was collected and each skin sample was wiped two times with Whatman filter paper soaked with PBS/Tween 20 and once with 70%/30% ethanol/water to remove unabsorbed (residual) product. The epidermis was separated from the dermis by dissection with forceps. The skin sections, epidermis and dermis, were separately dissolved in 0.50-1 mL Soluene-350 (Perkin Elmer, Boston, Mass.) at 50° C. overnight. Active materials in receptor phase, filter paper wipes and solubilized skin samples were quantified using liquid scintillation. For formulations excipients (cyclopentasiloxane and isohexadecane) that required the use of non-radioactive detection, analytical procedures are described in the Appendix.

To normalize the skin penetration data to mass balance, disintegrations-per-minute (DPMs) for each compartment of each diffusion cell were blank corrected and summed to obtain a total recovered radiolabel value for each cell. The DPMs of each compartment were then normalized to the total recovered radiolabel value to obtain a % of applied dose mass balance-normalized for each compartment (surface wipes, epidermis, dermis and receptor). Total permeated value represents the sum of epidermis, dermis and receptor.

From the results shown in FIG. 6, it can be seen that there is a significant increase in penetration into skin of inositol, particularly in the epidermis compared with either finger application or application via an applicator incorporating a magnet.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application and any patent application or patent to which this application claims priority or benefit thereof, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. An applicator for a skin care product, comprising:
a) a substrate with a magnetic array embedded therein, said magnetic array having at least one layer of one or more dipolar pairs of alternating magnetic poles; and
b) a vibration source.

2. The applicator of claim 1, wherein the magnetic poles are located a distance x (mm) apart, wherein x equals 1 or more.

3. The applicator of claim 2, wherein the vibration source is configured to vibrate with an amplitude of vibration at least half of x.

4. The applicator of claim 1, wherein the vibration source is configured to vibrate with an amplitude of up to 2 mm.

5. The applicator of claim 1, wherein the vibration source has a frequency of vibration of between 0.5 Hz to 1000 Hz.

6. The applicator of claim 1, wherein the vibration source is a motor driving an off-center fly-weight and a speed of rotation of between 2500 and 10000 RPM.

7. The applicator of claim 1, further comprising:
a) an electrically conductive tip;
b) a handle having an electrically conductive base; and
c) a power source for the vibration source, wherein the electrically conductive tip and electrically conductive base are part of a touch switch circuit that activates the power source upon detecting a current path.

8. The applicator of claim 1, further comprising:
a) a power source for the vibration source;
b) a tip for making contact with a user's skin; and
c) one or more sensors provided in the tip for activating the power source upon contact of the applicator with skin.

9. The applicator of claim 1, wherein the magnetic array comprises a first layer of at least one dipole pair of alternating magnetic poles with a pitch of between 1.7 and 2.5 and a magnetic field strength of between about 24.0 and 36.0 mT.

10. The applicator of claim 9, wherein the first layer has a thickness of between 0.8 and 1.2 mm.

11. The applicator of claim 9, wherein the magnetic array is optimized for use together with a skin care active selected from the group comprising: a vitamin B3 active, preferably niacinamide; a peptide, preferably Pal KTTKS; or a sugar alcohol, preferably inositol.

12. A cosmetic skin care product, comprising:
a) the applicator of claim 1; and
b) a skin care composition comprising a skin care active selected from palmitoyl-lysine-threonine-threonine-lysine-serine peptide, a vitamin B3 compound, inositol, and combinations thereof, and a dermatologically acceptable carrier.

13. A method of cosmetically treating a skin condition, comprising:
a) identifying a target portion of skin where treatment is needed or desired; and
b) applying a skin care composition to the target portion of skin with the applicator of claim 1.
